(19) United States
(12) Patent Application Publication
(10) Pub. No.: US 2016/0046665 A1
(43) Pub. Date: Feb. 18, 2016

(54) Peptide Ligand with Antibody Selectivity and the Application Thereof

(71) Applicant: National Central University, Taoyuan City (TW)

(72) Inventors: Ruoh-Chyu Ruaan, Taipei City (TW); Ching-Wei Tsai, Taoyuan County (TW); Siang-Long Jheng, New Taipei City (TW); Wei-Chih Tseng, Keelung City (TW); Wen-Yih Chen, Taoyuan County (TW)

(21) Appl. No.: 14/624,223

(22) Filed: Feb. 17, 2015

(30) Foreign Application Priority Data
Aug. 18, 2014 (TW) 103128353

(51) Int. Cl. C07K 1/22; C07K 16/00; C07K 5/113; C07K 7/06; C07K 5/11

(52) U.S. Cl. CPC C07K 1/22; C07K 7/06; C07K 5/1019; C07K 5/1021; C07K 16/00

(57) ABSTRACT

This invention discloses a peptide ligand with antibody selectivity and the application thereof. The peptide ligand with antibody selectivity comprises peptide ligand consisted of a sequence with 4 to 6 amino acids. The peptide ligand with antibody selectivity is able to bind with the hydrophobic region at the bottom of antibody's Fc region through non-covalent bonding. The mentioned peptide ligand with antibody selectivity can be applied to biochip for antibody oriented immobilization, and the biochip can provide high recognition efficiency to antigen. Besides, the mentioned peptide ligand with antibody selectivity can be applied as antibody purification material for purifying antibody with respective peptide ligand with antibody selectivity.

### BACKGROUND OF THE INVENTION

1. Field of the Invention

The present invention is generally related to a peptide ligand, and more particularly to a peptide ligand with antibody selectivity and antibody oriented immobilization on solid supports or substrate, and the application thereof.

2. Description of the Prior Art

Human beings and animals have their own inherent immune system. Theoretically, if the immune system works efficiently, all diseases, including serious diseases as diabetes mellitus and cancer, can be overcome. Antibody, known as Immunoblobulin (Ig), is secreted by B lymph cell, and is used for identifying and neutralizing extrinsic materials, such as large Y-shaped protein pathogen. Antibody can identify one specific material of the extrinsic material, and the specific material is named antigen. There is a lock-shaped region, so-called paratope (epitope), on each forked top of they-shaped protein. The mentioned lock-shaped region is for a specific antigen.

Antigen and immunogen both can induce acquired immune response while receiving exotic stimulation, but the functions of antigen and immunogen are different. Antigen is material, including lymphocyte and antibody, being able to specifically engage with immune system. Immunogen is material being able to induce immune response.

On manufacturing immune detective biochip or immune related bio device, the immobilized way of antibody plays an important role for identification efficiency of antibody and antigen. So that antibody oriented immobilization is an indispensable step during developing immune relative technology. The definition of “immobilization” is fixing bio-molecules on a substrate surface, and the fixed bio-molecules will lose part of activity. When fixing antibody on a substrate surface, part activity of the fixed antibody will be lost, and the bio-activity of the fixed antibody will be lowered. In recently years, in order to increasing the detective efficiency of immune biochip, there are many teams devoting to finding out highly stable immobilizing way and modifying the antibody orientation on surface.

The immobilizing methods of antibody can be roughly classified as the following: (1) covalent coupling, (2) physical adsorption, and (3) bio-affinity. It is very difficult to find out a proper method for immobilizing antibody. Moreover, in those recently immobilizing way of antibody, there are many problems, such as that the bio-activity of the immobilized antibody is decreased, or that the manufacturing cost of the device with immobilized antibody is too expensive.

In view of the above matter, developing a novel peptide ligand with antibody selectivity and the application thereof having high antigen detecting efficiency and having antibody oriented immobilization without effecting the recognition efficiency to antigen is still an important task for the industry.

### SUMMARY OF THE INVENTION

In light of the above background, in order to fulfill the requirements of the industry, the present invention provides a novel peptide ligand with antibody selectivity and the application thereof. The mentioned peptide ligand with antibody selectivity and the application thereof does not cost expensive, and can provide excellent property on antibody selectivity and antibody oriented immobilization. Preferably, the mentioned peptide ligand with antibody selectivity can be applied on immunology tools, such as detective bio-chip, and antibody purification. More preferably, after binding the mentioned peptide ligand with medicine or genetic material, the peptide ligand of this specification can be applied on medicine or genetic material transportation by the affinity between the peptide ligand and antibody. Therefore, the mentioned peptide ligand with antibody selectivity the application thereof are non-expensive and with high accuracy, and can be very useful for advancing industrial competitive.

One object of the present invention is to provide a peptide ligand with antibody selectivity and the application thereof, through employing a short chain peptide consisted of 4 to 6 amino acids, the synthesis program of peptide ligand can be simplified, and the manufacturing cost thereof can be decreased.

Another object of the present invention is to provide peptide ligand with antibody selectivity and the application thereof, through employing peptide ligand with proper chain length, hydrophobicity, and electric property, the affinity between the peptide ligand and the bottom hydrophobic region of antibody, so that the mentioned peptide ligand can provide excellent antibody oriented immobilization.

Still another object of the present invention is to provide peptide ligand with antibody selectivity and the application thereof, through employing an antibody oriented immobilization device with peptide ligand with antibody selectivity, it can be efficiently for achieving antibody oriented immobilization and antigen recognition.

Still another object of the present invention is to provide peptide ligand with antibody selectivity and the application thereof, through employing an antibody purification material with peptide ligand with antibody selectivity, it can be efficiently for isolating/purifying target antibody.

Accordingly, the present invention discloses a peptide ligand with antibody selectivity and the application thereof. The peptide ligand with antibody selectivity comprises peptide ligand consisted of 4 to 6 amino acids. The mentioned peptide ligand has a hydrophilic end and a hydrophobic end. The peptide ligand can bind to a fragment crystallizable (Fc) region of an antibody through non-covalent bonding.

In one preferred example of this specification, the mentioned peptide ligand can be selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

In one preferred example, the mentioned peptide ligand can bind/embed with the bottom hydrophobic region of an antibody through hydrophobic interaction and electrostatic force.

In one preferred example of this specification, the mentioned peptide ligand with antibody selectivity can be applied to antibody oriented immobilization device. The mentioned antibody oriented immobilization device comprises substrate, and a plurality of peptide ligand with antibody selectivity fixed on the surface of the substrate. According to this example, through those peptide ligand with antibody selectivity, antibody can be oriented immobilized to the substrate surface, and then the substrate can provide excellent antigen recognition.

In one preferred example of this specification, the mentioned peptide ligand with antibody selectivity can be applied to antibody purification material. The mentioned antibody purification material comprises a plurality of media and a plurality of peptide ligand with antibody selectivity. There is at least one peptide ligand with antibody selectivity fixed on each of the media. According to this example, through selecting respective peptide ligand, it can be efficiently achieving target antibody isolation/purification.

### BRIEF DESCRIPTION OF THE DRAWINGS

FIG. 1A shows the absorption SPR signal spectrum of Rabbit IgG and PSA on different chips, wherein (A) is chip with positive charge surface, (B) is chip with negative charge surface, (C) is chip with EGELW surface, and (D) is chip with RRGW surface;

FIG. 1B shows the absorption of Rabbit IgG on different chip surfaces in FIG. 1A;

FIG. 2 shows the absorption of Mouse IgG₂ₐ on different chip surfaces;

FIG. 3A shows the absorption of PSA on different chip surfaces with Rabbit IgG;

FIG. 3B shows the absorption of PSA on different chip surfaces with Mouse IgG₂ₐ;

FIG. 4A shows the antigen recognition of PSA on different chip surfaces with Rabbit IgG;

FIG. 4B shows the antigen recognition of PSA on different chip surfaces with Mouse IgG₂ₐ;

FIG. 5A shows the absorption SPR signal spectrum of Rabbit IgG and 2^{nd} antibody on different chip surfaces, wherein (A) is chip with positive charge surface, (B) is chip with negative charge surface, (C) is chip with EGELW surface, and (D) is chip with RRGW surface;

FIG. 5B shows the absorption of 2^{nd} antibody on different chip surfaces with Rabbit IgG;

FIG. 5C shows the absorption of 2^{nd} antibody on different chip surfaces with Mouse IgG₂ₐ;

FIG. 6A shows the orientation factors of different chip surfaces with Rabbit IgG;

FIG. 6B shows the orientation factors of different chip surfaces with Mouse IgG₂ₐ;

FIG. 7A shows the SPR sensitivity detecting results of different chip surfaces with Rabbit IgG;

FIG. 7B shows the SPR sensitivity detecting results of different chip surfaces with Mouse IgG₂ₐ; and

FIG. 8 shows the affinity constants of Bovine serum albumin (BSA), Rabbit IgG, and Mouse IgG₂ₐ respectively absorbed on CM-Sepharose, EGELW-CM-Sepharose, and RRGW-CM-Sepharose.

### DESCRIPTION OF THE PREFERRED EMBODIMENTS

What probed into the invention is peptide ligand with antibody selectivity and the application thereof. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment according to this specification discloses a peptide ligand with antibody selectivity. The peptide ligand with antibody selectivity comprises peptide ligand consisted of sequence with 4 to 6 amino acids. According to this embodiment, the peptide ligand has a hydrophilic end and a hydrophobic end. The peptide ligand can bind with a fragment crystallizable (Fc) region of an antibody through non-covalent bonding.

In one preferred example of this embodiment, the mentioned peptide ligand can be selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

In one preferred example of this embodiment, the mentioned peptide ligand can bind/embed with the bottom region of an antibody through hydrophobic interaction and electrostatic force.

Another preferred embodiment according to this specification discloses an antibody oriented immobilization device with peptide ligand with antibody selectivity. The mentioned antibody oriented immobilization device comprises substrate, and a plurality of peptide ligand with antibody selectivity fixed on the surface of the substrate. According to this embodiment, the peptide ligand comprises a peptide consisted of a sequence with 4 to 6 amino acids. Each of the peptide ligands has a hydrophilic end and a hydrophobic end. The peptide ligands can bind with a fragment crystallizable (Fc) region of an antibody through non-covalent bonding, so that the antibody can be oriented immobilized on the substrate. In one preferred example of this embodiment, the mentioned peptide ligands can be fixed on the surface of the substrate through covalent bonding.

In one preferred example of this embodiment, the mentioned peptide ligands can be selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

In one preferred example of this embodiment, the mentioned peptide ligand can bind/embed with the bottom region of an antibody through hydrophobic interaction and electrostatic force. In one preferred example of this embodiment, the mentioned peptide ligand can bind/embed with the bottom hydrophobic region of an antibody through hydrophobic interaction and electrostatic force.

In one preferred example of this embodiment, the substrate is selected from the group consisting of the following: Au, SiO₂, silicon wafer, Fe₃O₄, Fe₂O₃, and organic polymer.

Still another preferred embodiment according to this specification discloses an antibody purification material with peptide ligand with antibody selectivity. The mentioned antibody purification material comprises a plurality of media, and a plurality of peptide ligand with antibody selectivity fixed on the surface of the media. According to this embodiment, the peptide ligand comprises peptide ligand consisted of a sequence with 4 to 6 amino acids. Each of the peptide ligands has a hydrophilic end and a hydrophobic end. The peptide ligands can bind with a fragment crystallizable (Fc) region of an antibody through non-covalent bonding.

In one preferred example of this embodiment, the mentioned peptide ligands can be selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

In one preferred example of this embodiment, the mentioned peptide ligand can bind/embed with the bottom hydrophobic region of an antibody through hydrophobic interaction and electrostatic force.

In one preferred example of this embodiment, the media can be selected from the group consisting of the following: bead, particle, membrane, semi-permeable membrane, capillary, microarray, multiple well plate, glass plate, silicon wafer, and tissue culture plate.

In one preferred example of this embodiment, the media can be in-organic material. The mentioned in-organic material can be selected from at least one of the group consisting of the following: glass, alumina, silica, zirconia, magnetite, and semiconductor.

In one preferred example of this embodiment, the media can be consisted of organic material. The mentioned organic material can be selected from at least one of the group consisting of the following: polysaccharides, polyacrylamide, polyacrylate, polystyrene, and polyvinyl alcohol. The mentioned polysaccharides can be selected from at least one of the group consisting of the following: agarose, dextran, cellulose, chitosan, and sepharose.

In one preferred example of this embodiment, the mentioned antibody purification material with peptide ligand with antibody selectivity can be fixed on a substrate through physical or chemical way. The mentioned substrate can be selected from the group consisting of the following: permeable membrane, semi-permeable membrane, glass plate, gold, tissue culture plate, silicon wafer, and polymer.

In another preferred example of this embodiment, the mentioned antibody purification material with peptide ligand with antibody selectivity can be packed in a column. The antibody purification material with peptide ligand with antibody selectivity can be used to purify/isolate antibody by chromatography.

For demonstrating, the following will disclose several examples and tests of peptide ligand with antibody selectivity and the application thereof according to this invention. It is noted that these examples are not to limit the scope of this present invention, which should be determined in accordance with the Claims.

### Example 1

### Preparation of SPR Chip Surface Modification

In this and the following examples, we employ Surface Plasmon resonance (SPR) as detecting equipment. The method for modifying SPR chip surface is briefly illustrated as following.

OEG [(11-Mercaptoundecyl)tetra(ethyleneglycol)] and OEG-COOH [HS(CH₂)₁₁(EG)₆OCH₂COOH] are mixed at the ratio of 10:1. Through strong gold-sulfur bonding, OEG and OEG-COOH are employed for modifying SPR chip surface to form mixed SAM (self-assembly monolayer). The mixed SAM surface comprises carboxyl group with negative charge, and hydroxyl group with hydrophilicity. The carboxyl group with negative charge on the SPR chip surface can be activated by EDC/20% DMSO. By reacting the 1° amino group of EDA (ethylenediamine) with the carboxyl group on the SPR chip surface to form amide bonding, the SPR chip surface can be modified into positive charge. And, in the same condition, the peptide ligand according to this invention can be grafted onto the SPR chip surface to form a peptide ligand surface.)

Mixed SAM: Surface Modification of SPR Chip with Negative Charge

1. The copper-Lovchorrite at the surface of SPR gold chip is wiped by non-dust cloth with few acetone.

2. The gold chip is washed with 95% ethanol and ultrapure water for 3 times in sequence.

3. The gold chip surface is cleaned by UV ozone cleaning system for 20 minutes.

4. The gold chip is washed with 95% ethanol and ultrapure water for 3 times in sequence.

5. OEG and OEG-COOH are mixed at the ratio of 10:1 for preparing 1 mM thiol solution in 4 mL 95% ethanol.

6. The gold chip and the mentioned solution are put into a Teflon box, and reacted for 16 hours at 40° C.

7. The gold chip is taken out, and washed with 10% NH₄OH and ethanol.

8. After washed with ultrapure water, the gold chip is kept in 4° C. ultrapure water.

Mixed SAM: Surface Modification of SPR Chip with Positive Charge

1. 5 mM EDA, 200 mM EDC, and 20% DMSO are prepared into 0.1 M pH=6.0 MES buffer solution.

2. The mentioned buffer solution is added to gold chip surface, and reacted for 4 hours at 4° C.

3. The gold chip is taken out and washed with ultrapure water.

4. 10 mM ethanolamine (ETA) solution is prepared in 0.1 M pH=6.0 MES buffer solution.

5. The mentioned ETA solution is added to the gold chip surface, and reacted for 2 hours at 25° C.

6. After washed with ultrapure water, the gold chip is kept in 4° C. ultrapure water.

Mixed SAM: Surface Modification of SPR Chip with Peptide Ligand

1. 5 mM peptide ligand, 200 mM EDC, and 20% DMSO are prepared into 0.1 M pH=6.0 MES buffer solution.

2. The mentioned buffer solution is added to gold chip surface, and reacted for 4 hours at 4° C.

3. The gold chip is taken out and washed with ultrapure water.

4. 10 mM ethanolamine (ETA) solution is prepared in 0.1 M pH=6.0 MES buffer solution.

5. The mentioned ETA solution is added to the gold chip surface, and reacted for 2 hours at 25° C.

6. After washed with ultrapure water, the gold chip is kept in 4° C. ultrapure water.

### Example 2

### Analysis of Antigen Recognition Efficiency

In this example, SPR is employed for measuring the absorption of target antibody, rabbit immunoglobulin (Rabbit IgG), and target antigen, prostate specific antigen (PSA), on different charge surfaces and different peptide ligand surfaces. The mentioned different chip surfaces are employed for absorbing Rabbit IgG, washed with phosphate buffer (PB) solution, and employed to absorb PSA directly. Then, PB buffer solution is used to wash out the PSA not recognized by Rabbit IgG for measuring the absorption of antibody and antigen. The experimental process is roughly shown as following.

1. 10 μg/mL Rabbit IgG is prepared in 0.01 M pH=7.4 PB buffer solution.

2. 5 μg/mL PSA is prepared in 0.01 M pH=7.4 PB buffer solution.

3. The negative charge, positive charge, and peptide ligand modified chips are put on SPR respectively.

4. The flow rate is set at 20 μL/min.

5. Rabbit IgG, PB, PSA, and PB are flowed through the channel in sequence. The absorption time is about 60 minutes, and the desorption time is about 50 minutes.

### Example 3

### Analysis of Antibody Oriented Immobilization

In this example, SPR is employed for the oriented immobilization analysis of Rabbit IgG, and Secondary antibody (2^{nd} antibody; 2^{nd} Ab) is used to replace PSA in Example 2 to detect the absorption of Rabbit IgG, and 2^{nd} Ab. The experimental process is roughly shown as following.

1. 10 μg/mL Rabbit IgG is prepared in 0.01 M pH=7.4 PB buffer solution.

2. Goat anti-Rabbit IgG 10 μg/mL (2^{nd} Ab) is prepared in 0.01 M pH=7.4 PB buffer solution.

3. The negative charge, positive charge, and peptide ligand modified chips are put on SPR respectively.

4. The flow rate is set at 20 μL/min.

5. Rabbit IgG, PB, 2^{nd} Ab, and PB are flowed through the channel in sequence. The absorption time is about 60 minutes, and the desorption time is about 50 minutes.

### Example 4

### Processing Antibody Purification with Peptide Ligand Grafted on CM-Sepharose by Covalent Bond

10 mL CM-Sepharose (the concentration of the COOH ligand is 130 mM) is washed with 100 mL deionized water, and centrifuged at 3000 rpm. After removing the supernatant fluid, 10 mL EDC/NHS (26 mM:52 mM) dissolved in PBS solution is added to the CM-Sepharose, and shacked for 30 minutes at 37° C. The mixed of the CM-Sepharose, EDC, and NHS solution is centrifuged at 3000 rpm. After removing the supernatant fluid, 10 mL PBS solution with 13 mM EGELW peptide, or 13 mM RRGW peptide is added to the CM-Sepharose. After reacting for 1 day, it can be obtained EGELW-CM-Sepharose or RRGW-CM-Sepharose.

After packing 5 mL EGELW-CM-Sepharose or RRGW-CM-Sepharose into a chromatographic column, the mixture of Bovine serum albumin (BSA) and antibody (mouse IgG₂ₐ or Rabbit IgG) is loaded to the chromatographic column for purification/isolation with HPLC, wherein the mobile phase A in HPLC is 10 mM pH 7.4 phosphoric acid, and mobile phase B is pH 7.4 PBS buffer solution. The flow rate is 0.5 mL/min of isocratic elution with mobile phase A for 15 minutes and then gradient elution with mobile phase A to mobile phase B for 30 minutes.

When employing EGELW-CM-Sepharose as the stationary phase in the column, the retention time of BSA is 7.2 minutes, the retention time of mouse IgG₂ₐ is 14.8 minutes, and the retention time of Rabbit IgG is 28.5 minutes.

When employing RRGW-CM-Sepharose as the stationary phase, the retention time of BSA is 9.2 minutes, the retention time of mouse IgG₂ₐ is 16.8 minutes, and the retention time of Rabbit IgG is 20.0 minutes.

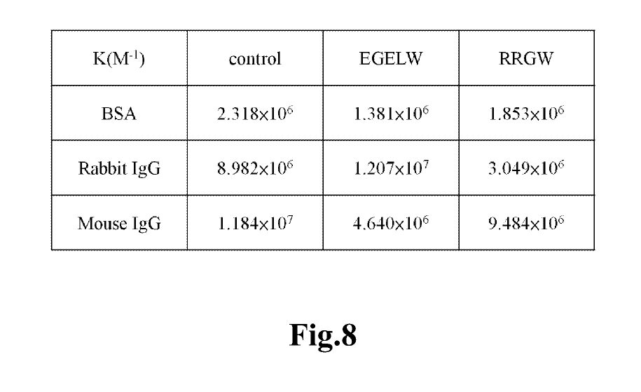

The adsorption affinity constants of BSA and antibody (mouse IgG₂ₐ or Rabbit IgG) and those three stationary phases (CM-Sepharose, EGELW-CM-Sepharose, RRGW-CM-Sepharose) are shown in FIG. 8.

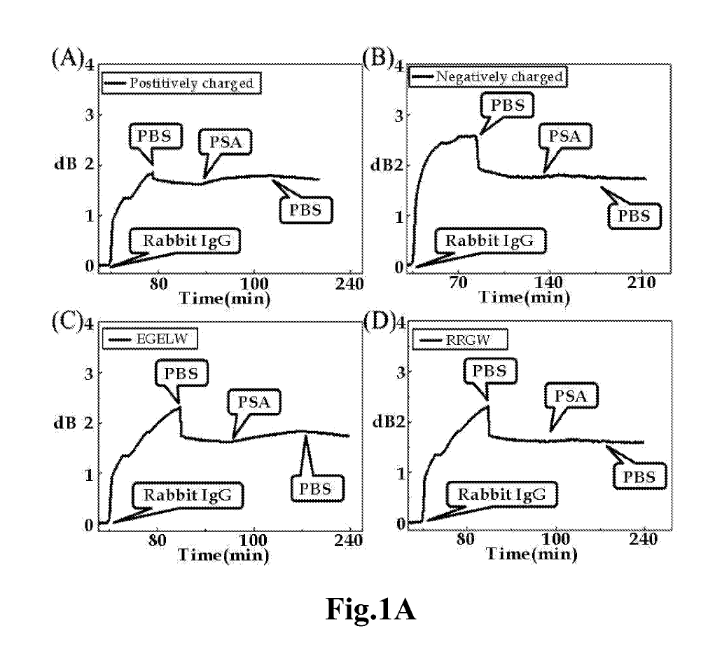

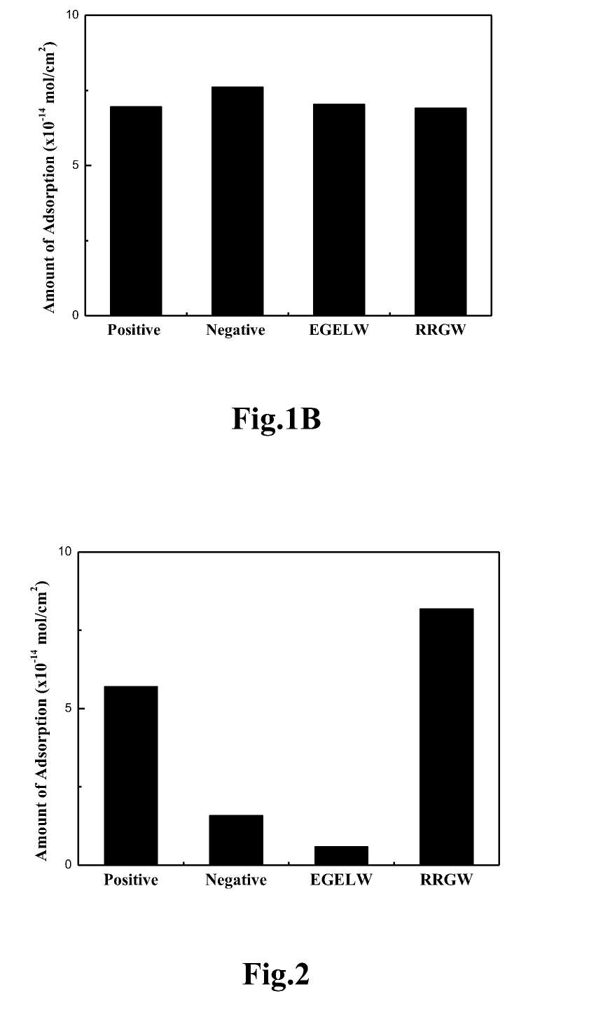

In the above experiments, Surface Plasmon Resonance (SPR) is employed for measuring the antigen recognition efficiency of antibody on different modified chip surfaces. The recognition efficiency of Rabbit IgG and PSA on different chip with different charge surface and different peptide ligand is detected. FIG. 1A shows the absorption signals of Rabbit IgG and PSA on four kinds of chip surfaces VS time. After transferring those signals in FIG. 1A to protein absorption amounts through the following formula, FIG. 1B can be further obtained.

$$Δ 1 \times \frac{1}{Δ 2} \times 4.68 \times {10}^{- 9} \div M . W . = \_mol \text{/} {\text{cm}}^{2}$$

In the above formula, Δ1 shows the signal difference of protein desorption, Δ1 shows the solvent background value of PB, M.W. shows protein molecular weight.

From FIG. 1B, it can be found that a large number of antigens can be absorbed by all Rabbit IgG on those four different chip surfaces. Particularly, it can be noticed that the absorption on the negative charge chip surface is about 8.52 mol/cm². During looking for the ideal location in Fc region for binding Rabbit IgG, we found that there is almost all positive charge surrounding the ideal binding location, so that the absorption on the negative charge chip surface will be increased. It also can be found that the absorption on the positive charge chip surface is about 6.55 mol/cm². The absorption of those two different charge chip surfaces is not much difference. Accordingly, it can be deduced that the surface charge is uniformly distributed on the surface of Rabbit IgG, so that a large of absorption can be measured on both the chip surfaces with different charge. Besides, from FIG. 1B, it can be found that there is not much difference between the Rabbit IgG absorption on those chip surfaces modified with different peptide ligands as EGELW and RRGW. For further researching, the absorption of Mouse IgG₂ₐ on different chip surfaces is shown as FIG. 2.

Comparing FIG. 1B and FIG. 2, it can be found that the absorption of RRGW to these antibody are both great. That is, RRGW does not have specificity to these antibodies. It also can be found that there is a large number of Rabbit IgG absorbed by EGELW, and there is few Mouse IgG₂ₐ absorbed by EGELW. That presents the specificity of EGELW to these antibodies.

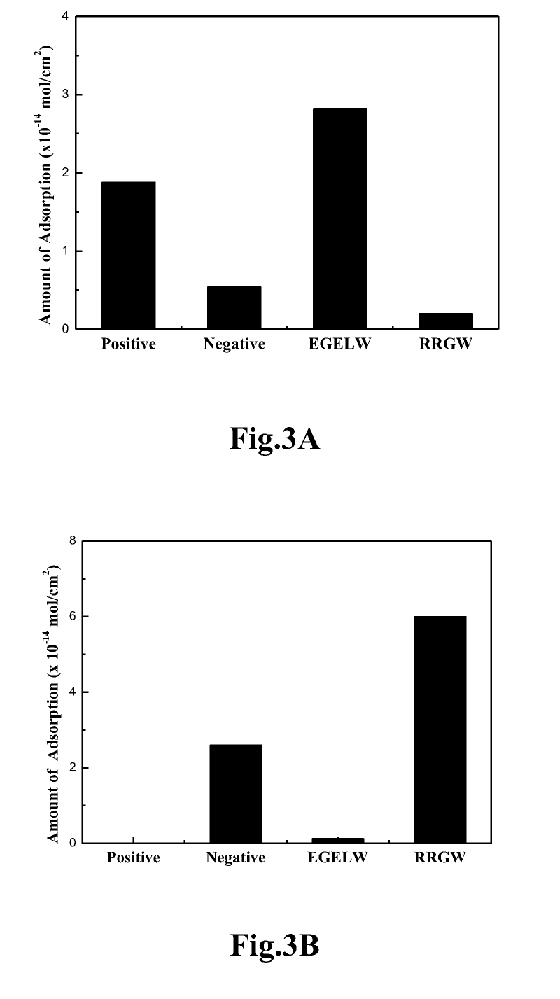

Different chips with Rabbit IgG and Mouse IgG₂ₐ are used for PSA absorption experiment, and the PSA absorption is transferred to protein absorption by the above-mentioned formula, as shown in FIG. 3A and FIG. 3B.

From FIG. 3A and FIG. 3B, it can be found that the Rabbit IgG absorption to the negative charge chip surface is about 8.52 mol/cm², and the PSA absorption to the negative charge chip surface is about 0.538 mol/cm². Accordingly, it can be deduced that the oriented immobilization of Rabbit IgG on negative charged chip surface is poor. There is not much difference between the Rabbit IgG absorption to those two different peptide ligand chip surfaces respectively with EGELW and RRGW, and there are differences between the PSA absorption to those two different peptide ligand chip surfaces respectively with EGELW and RRGW. The Rabbit IgG absorption to the chip surface with EGELW is about 2.82 mol/cm², and the Rabbit IgG absorption to the chip surface with RRGW is just about 0.2 mol/cm². Additionally, Rabbit IgG can provide better oriented immobilization to EGELW chip surface. From FIG. 3A and FIG. 3B, it can be found that there are quiet differences between the Mouse IgG₂ₐ absorption to those two different peptide ligand chip surfaces respectively with EGELW and RRGW, and the Mouse IgG₂ₐ absorption to the peptide ligand chip surface with RRGW is higher. Similarly, the PSA absorption to the peptide ligand chip surface with RRGW is great. Consequently, it can be deduced that the oriented immobilization of Mouse IgG₂ₐ on RRGW peptide ligand chip surface is great.

In order to compare antigen recognition efficiency, we define “antigen recognition efficiency” as following:

$$antigen recognition efficiency = \frac{PSA absorption ( mol \text{/} {cm}^{2} )}{Antibody absorption ( mol \text{/} {cm}^{2} )}$$

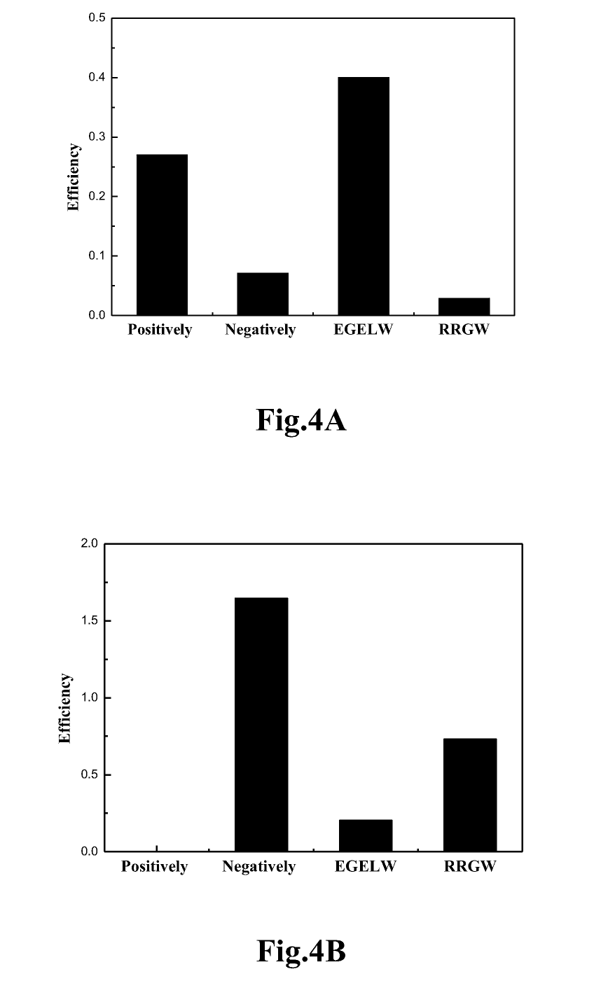

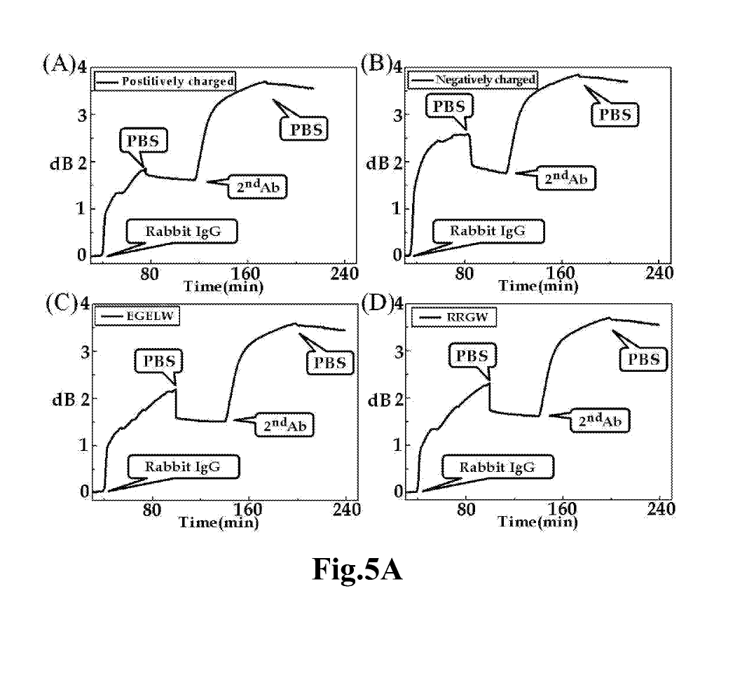

According to the above formula of antigen recognition efficiency, FIG. 3A and FIG. 3B can be transferred to FIG. 4A and FIG. 4B.

From FIG. 4A and FIG. 4B, it can be found that the antigen recognition efficiency of Rabbit IgG is great on the peptide ligand chip surface with EGELW, and the antigen recognition efficiency of Rabbit IgG is not good on the peptide ligand chip surface with RRGW. Relatively, the antigen recognition efficiency of Mouse IgG₂ₐ is great on the peptide ligand chip surface with RRGW, and the antigen recognition efficiency of Mouse IgG₂ₐ is not good on the peptide ligand chip surface with EGELW. Therefore, it can be deduced that peptide ligand RRGW is helpful for the oriented antigen recognition of Mouse IgG₂ₐ to PSA, and peptide ligand EGELW is helpful for the oriented antigen recognition of Rabbit IgG to PSA.

Moreover, for analyzing the orientation of Rabbit IgG on the chip surface, 2^{nd} antibody being able to recognize the Fc region of Rabbit IgG is employed in following experiment. If the Fc region of Rabbit IgG on the chip surface is exposed to the solution, the Fc region of Rabbit IgG will be recognized by the 2^{nd} antibody. That points out that the Rabbit IgG is fixed on the chip surface in wrong orientation.

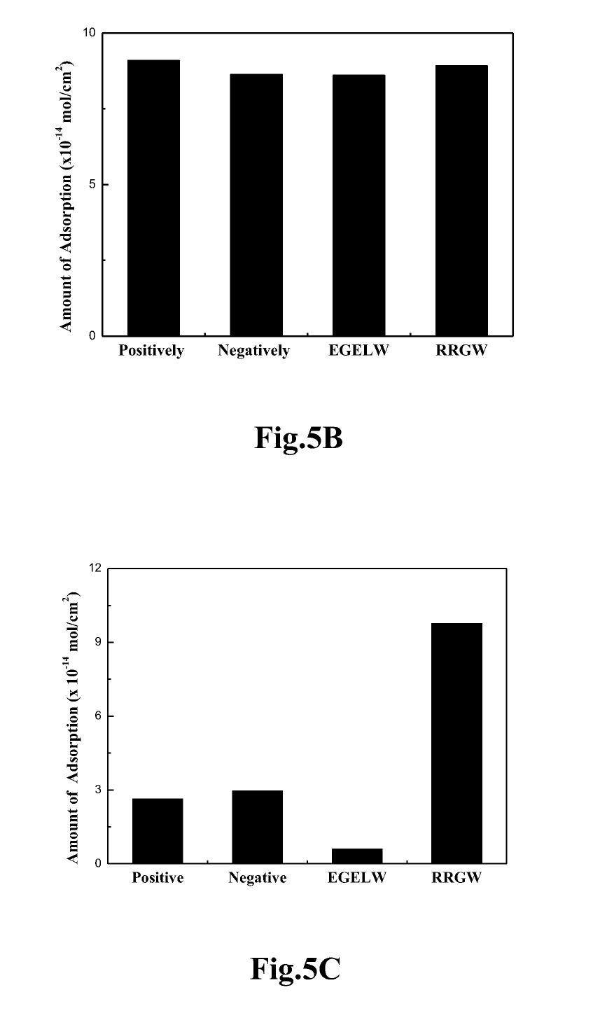

Experimentally, Surface Plasmon Resonance (SPR) is employed for measuring the 2^{nd} antibody absorption on different chip surfaces for comparing the orientation of the Rabbit IgG and the 2^{nd} antibody on different chip surfaces. FIG. 5A presents the figure of the absorption signals of Rabbit IgG and 2^{nd} antibody on four different chip surfaces VS time.

Similarly, through using the above-mentioned formula to transfer those absorption signals in FIG. 5A into protein absorption, FIG. 5B is obtained. And, the absorption of Rabbit IgG and 2^{nd} antibody on four different chip surfaces is shown in FIG. 5C.

From FIG. 5B, the absorption of 2^{nd} antibody is great on those different chip surfaces with Rabbit IgG, especially on the chip surface with peptide ligand. It is suggested that there is still some Rabbit IgG fixed on the chip surface in wrong orientation. So, excluding the absorption of 2^{nd} antibody, the ratio of PSA and 2^{nd} antibody should also be used for checking the orientation difference. Hence, we define “orientation factor” as following.

$$orientation factor = \frac{PSA absorption ( mol \text{/} {cm}^{2} )}{2 nd Antibody absorption ( mol \text{/} {cm}^{2} )}$$

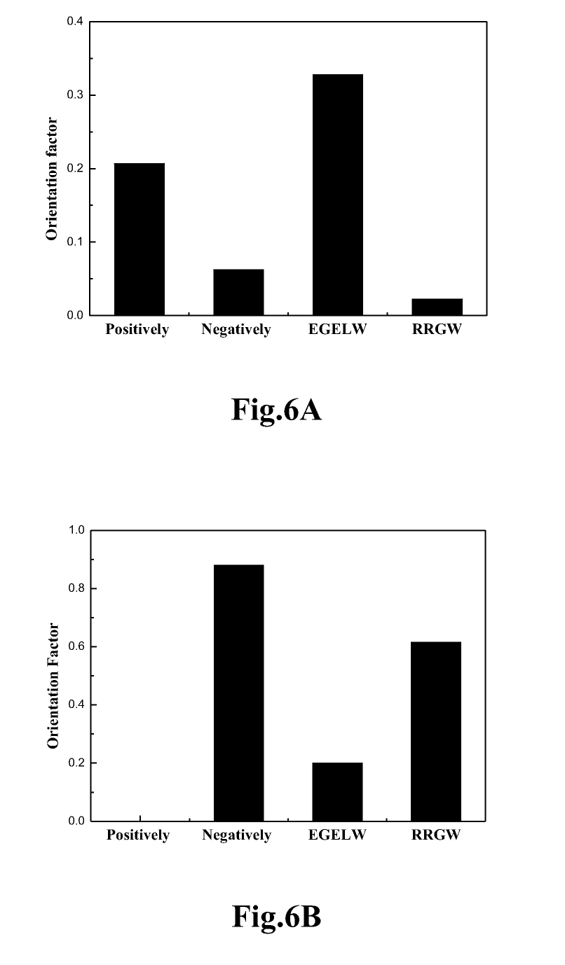

If the orientation factor is raised, there are more antibodies being able to be recognized by PSA on the chip surface, and less antibody being able to be recognized by 2^{nd} antibody on the chip surface. That is, we can use the orientation factor to compare the antibody orientation on different chip surfaces, as shown in FIG. 6A and FIG. 6B.

From FIG. 6A, it can be found that peptide ligand EGELW chip surface with Rabbit IgG presents higher orientation factor. It can be deduced that EGELW is helpful for presenting better orientation to Rabbit IgG. That is, the more PSA is cognized by Rabbit IgG on the peptide ligand EGELW chip surface; the less PSA is recognized by the 2^{nd} antibody. It also can be found that the positive charged chip surface is helpful to orientation factor. It can be deduced that PSA can be absorbed on positive charged chip surface, and the orientation factor is raised therefrom. From FIG. 6B, it can be found that peptide ligand RRGW chip surface with Mouse IgG₂ₐ presents good orientation factor. It can be deduced that RRGW is helpful for presenting better orientation to Mouse IgG₂ₐ.

Therefore, according to the mentioned peptide ligand designing and the above strategy screening, it can be found that peptide ligands, such as EGELW and RRGW, are useful for oriented immobilization of antibody, as Rabbit IgG and Mouse IgG₂ₐ, on chip surface, and are useful for increasing antigen recognition efficiency.

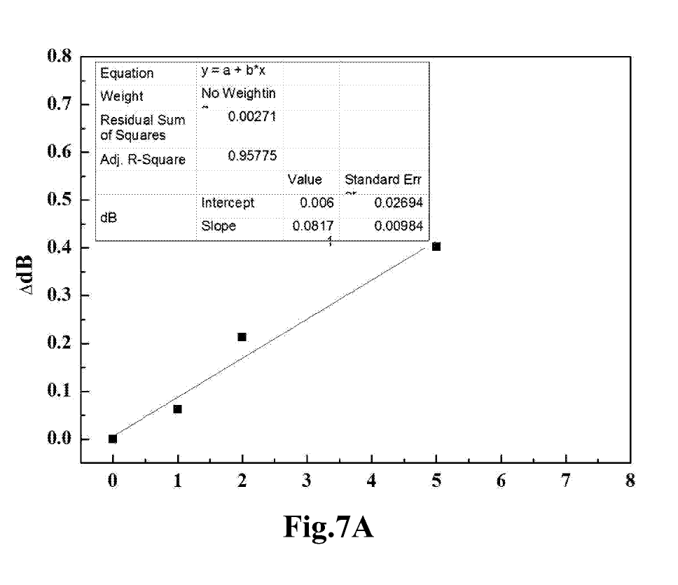

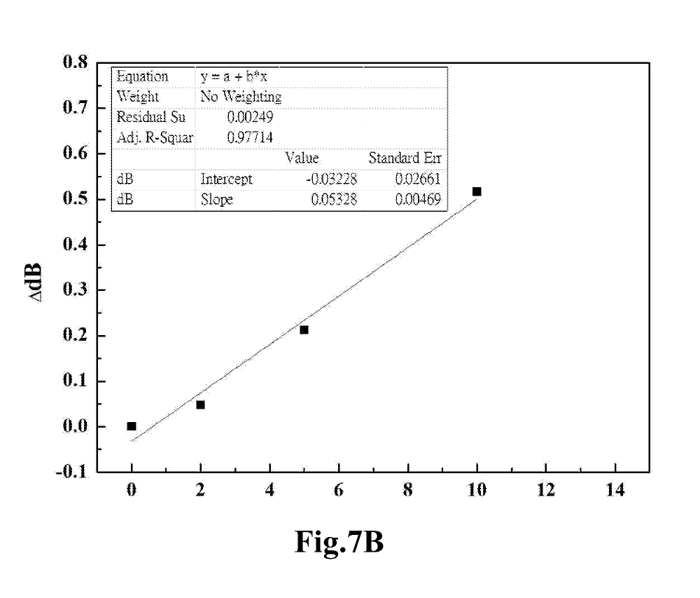

In the following experiments, antigen in micro concentration is employed for detecting the sensitivity of peptide ligand chips being able to recognize Rabbit IgG and Mouse IgG₂ₐ, and the experimental results are shown in FIG. 7A and FIG. 7B.

Firstly, Rabbit IgG and Mouse IgG₂ₐ are respectively absorbed to GGEGELW and GGRRGW. After flowing micro concentration antigen through, Rabbit IgG and Mouse IgG₂ₐ are flowed through the chip surface for increasing the detected signal. It can be found that both the signals from the mentioned peptide ligand chips can be detected by SPR in micro concentration antigen. According to FIG. 7A and FIG. 7B, the signal of about 2 ng/mL PSA can be detected from Mouse IgG₂ₐ of GGRRGW chip surface, and the signal of about 1 ng/mL PSA can be detected from Rabbit IgG of GGEGELW chip surface. Therefore, it proves that the peptide ligand of this specification can efficiently improve the antigen sensitivity.

For applying on purifying antigen with peptide ligand of this specification, referred to the above Example 4, after grafting peptide ligand to resin by covalent bonding, the resin is packed in chromatographic column for processing antigen purification/isolation. From the chromatographic experiment in Example 4, it can be found that the resin, grafted with peptide ligand with antibody selectivity through covalent bonding, does present great result in antibody purification/isolation. In other words, the peptide ligand of this specification can be applied as antibody purification material. According to this specification, including grafted to resin, it should be noticed that the peptide ligand of this specification also can be grafted to other substrate known by one skilled in that art, such as membrane, semi-permeable membrane, capillary, microarray, multiple well plate, glass plate, silicon wafer, and tissue culture plate, for obtaining antibody purification property.

In summary, this invention discloses peptide ligand with antibody selectivity and the application thereof. The peptide ligand with antibody selectivity comprises peptide ligand consisted of a sequence with 4 to 6 amino acids. The mentioned peptide ligand is able to bind with/embed to the hydrophobic region at the bottom of antibody's Fc region through non-covalent bonding. According to this specification, the peptide ligand with antibody selectivity can be applied to device for antibody oriented immobilization. The mentioned antibody oriented immobilization device with peptide ligand with antibody selectivity comprises substrate, and a plurality of peptide ligand with antibody selectivity fixed on the surface of the substrate. Through the peptide ligand with antibody selectivity, the mentioned antibody oriented immobilization device can efficiently oriented immobilizing antibody on the substrate surface, and can provide great antigen recognition efficiency. More preferably, the mentioned peptide ligand with antibody selectivity can be applied as antibody purification material. The mentioned antibody purification material comprises a plurality of media, and a plurality of peptide ligand with antibody selectivity. Each media is fixed at least one of the peptide ligand with antibody selectivity. Antibody can be purified through engaging the peptide ligand with antibody selectivity and the antibody, and subsequently desorbing the antibody from the peptide ligand with antibody selectivity. More preferably, according to this specification, the respective peptide ligand with antibody selectivity can be chosen to the target antibody while purifying antibody. According to this specification, the mentioned peptide ligand with antibody selectivity and the application thereof can be applied on antibody oriented immobilization, and can be applied on antibody purification/isolation. Therefore, this specification provides peptide ligand with antibody selectivity as a useful tool on immunology study, and the peptide ligand with antibody selectivity of this specification can be further applied on clinical immunology.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

1. A peptide ligand with antibody selectivity, comprising:
a peptide ligand consisted of a sequence with 4 to 6 amino acids, wherein said peptide ligand has a hydrophilic end and a hydrophobic end, wherein said peptide ligand can bind to a fragment crystallizable (Fc) region of an antibody through non-covalent bonding.

2. The peptide ligand with antibody selectivity according to claim 1, wherein said peptide ligand is selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

3. The peptide ligand with antibody selectivity according to claim 1, wherein said peptide ligand can bind to a bottom region of an antibody through hydrophobic interaction and electrostatic force.

4. An antibody oriented immobilization device with peptide ligand with antibody selectivity, comprising:
a substrate; and
a plurality of peptide ligand with antibody selectivity fixed on the surface of the substrate, wherein said peptide ligand with antibody selectivity comprises a peptide ligand consisted of a sequence with 4 to 6 amino acids, wherein said peptide ligand has a hydrophilic end and a hydrophobic end, wherein said peptide ligand can bind to a fragment crystallizable (Fc) region of an antibody through non-covalent bonding for oriented immobilizing the antibody on the substrate.

5. The antibody oriented immobilization device with peptide ligand with antibody selectivity according to claim 4, wherein said peptide ligand is selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

6. The antibody oriented immobilization device with peptide ligand with antibody selectivity according to claim 4, wherein said peptide ligand can bind to the bottom region of an antibody through hydrophobic interaction and electrostatic force.

7. The antibody oriented immobilization device with peptide ligand with antibody selectivity according to claim 4, wherein said substrate is selected from the group consisting of the following: Au, SiO₂, silicon wafer, Fe₃O₄, Fe₂O₃, and organic polymer.

8. An antibody purification material with peptide ligand with antibody selectivity, comprising:
a plurality of media; and
a plurality of peptide ligand with antibody selectivity wherein each of said media is fixed at least one of said peptide ligand with antibody selectivity, wherein said peptide ligand with antibody selectivity comprises a peptide ligand consisted of a sequence with 4 to 6 amino acids, wherein said peptide ligand has a hydrophilic end and a hydrophobic end, wherein said peptide ligand can bind to a fragment crystallizable (Fc) region of an antibody through non-covalent bonding.

9. The antibody purification material with peptide ligand with antibody selectivity according to claim 8, wherein said peptide ligand is selected from at least one of the group consisting of the following: EGEW, EEGW, EELW, RRGW, EGEGE, EGEGW, EGELW, EEGGW, EELLW, EELWL, EEWLW, EGEGW, EEGGLW, EGEGLW, RRGGLW, RGRGLW.

10. The antibody purification material with peptide ligand with antibody selectivity according to claim 8, wherein said peptide ligand can bind to the bottom hydrophobic region of an antibody through hydrophobic interaction and electrostatic force.

11. The antibody purification material with peptide ligand with antibody selectivity according to claim 8, wherein said media is selected from the group consisting of the following: bead, particle, membrane, semi-permeable membrane, capillary, microarray, multiple well plate, glass plate, silicon wafer, and tissue culture plate.

12. The antibody purification material with peptide ligand with antibody selectivity according to claim 8, wherein said media is consisted of in-organic material, wherein the in-organic material is selected from at least one of the group consisting of the following: glass, alumina, silica, zirconia, magnetite, and semiconductor.

13. The antibody purification material with peptide ligand with antibody selectivity according to claim 8, wherein said media is consisted of organic material, wherein the organic material is selected from at least one of the group consisting of the following: agarose, dextran, cellulose, chitosan, and sepharose.
